(19) United States
(12) Patent Application Publication
(10) Pub. No.: US 2025/0000929 A1
(43) Pub. Date: Jan. 2, 2025

(54) EVHIGH FLAVONOID CORN AND USES THEREOF

(71) Applicant: THE PENN STATE RESEARCH FOUNDATION, University Park, PA (US)

(72) Inventors: Surinder CHOPRA, University Park, PA (US); Alberto Gino LORENZONI CALVO, University Park, PA (US)

(21) Appl. No.: 18/708,993

(22) PCT Filed: Nov. 10, 2022
(86) PCT No.: PCT/US2022/049586

§ 371 Date: May 9, 2024

Related U.S. Application Data
(60) Provisional application No. 63/277,868, filed on Nov. 10, 2021.
(60) Provisional application No. 63/282,822, filed on Nov. 24, 2021.

(51) Int. Cl. A61K 36/899; A01H 5/10; A01H 6/46; A23K 10/22; A23K 10/30; A23K 20/158; A23K 50/75; A61P 1/00

(52) U.S. Cl. CPC A61K 36/899; A01H 5/10; A01H 6/4684; A23K 10/22; A23K 10/30; A23K 20/158; A23K 50/75; A61P 1/00

(57) ABSTRACT

The present invention provides the novel flavonoid-rich corn cultivar PennHFD. Animal feeds comprising kernels of this corn and methods of using the animal feeds to reduce necrotic enteritis and improve growth performance in animals are also provided.

### CROSS-REFERENCE TO RELATED APPLICATIONS

This application claims priority to U.S. Provisional Application No. 63/277,868 filed on Nov. 10, 2021, and to U.S. Provisional Application No. 63/282,822 filed on Nov. 24, 2021, the contents of which are incorporated by reference in their entireties.

### STATEMENT REGARDING FEDERALLY SPONSORED RESEARCH

This invention was made with government support under Grant No. 2011-67009-3017 awarded by the United States Department of Agriculture and under Hatch Act Project No. PEN04613 awarded by the United States Department of Agriculture/NIFA. The Government has certain rights in the invention.

### BACKGROUND

Avian necrotic enteritis (NE) is among the major diseases that affect the gastrointestinal health of broiler chickens (Kaldhusdal et al., 2016). It has been estimated that this disease could cost $6 billion worldwide every year due to reduced productivity and costs for treatment and prevention (Wade and Keyburn, 2015).

NE is caused by pathogenic strains of Clostridium perfringens, a spore-forming, anaerobic, Gram-positive bacterium that can be part of the native microbiota of humans and animals. Clinical NE develops in broiler chickens between two and five weeks of age, and causes depression, ruffled feathers, inappetence, and mortality. However, the subclinical form of NE (SNE) is more common, and it often goes undetected in commercial settings. SNE negatively impacts body weight gain and feed conversion ratio in chickens, claiming millions in revenue from producers around the world (Timbermont et al., 2011; Prescott et al., 2016a).

Several predisposing factors contribute to the development of NE, including high dietary non-starch polysaccharides and proteins, mycotoxins, and coccidiosis (Branton et al., 1987; Tsiouris et al., 2015; Broom, 2017). Coccidiosis is an intestinal tract infection caused by protozoan parasites from the genus Eimeria. Eimeria spp. disrupt the intestinal epithelium and create an optimal environment for the growth of C. perfringens, thereby contributing to the development of NE (Al-Sheikhly and Al-Saieg, 1980; Prescott et al., 2016b).

NE was effectively controlled when in-feed antibiotic growth promoters were widely used in animal production. However, the increasing concerns regarding antimicrobial resistance have led many countries to restrict the use of antibiotic growth promoters, which has led to increased outbreaks of NE (Kaldhusdal et al., 2016). Moreover, consumer preferences have shifted over the years towards antibiotic-free poultry products (Brewer and Rojas, 2007). Thus, there is a need in the art for alternative treatments for avian NE.

### SUMMARY

In a first aspect, the present invention provides the novel corn cultivar PennHFD (ATCC Accession No: 202111002). The invention encompasses the seeds of this cultivar, plants grown from these seeds, and seeds produced by these plants. The invention further encompasses any corn plant having all of the physiological and morphological characteristics of corn cultivar PennHFD.

In a second aspect, the present invention provides animal feeds comprising seeds of the flavonoid-rich corn described herein.

In a third aspect, the present invention provides methods for reducing the incidence of necrotic enteritis in a population of animals. The methods comprise administering corn seeds or an animal feed described herein to the animals.

In a fourth aspect, the present invention provides methods for improving the growth performance of an animal. The methods comprise administering corn seeds or an animal feed described herein to the animals.

In a fifth aspect, the present invention provides methods for producing flavonoid-rich corn plants. The methods comprise planting a plurality of PennHFD corn seeds under conditions favorable for the growth of corn plants. In some embodiments, the methods further comprise producing corn seeds from the resulting corn plants and/or planting the corn seeds.

### BRIEF DESCRIPTION OF THE DRAWINGS

FIG. 1 is a bar graph showing the incidence of intestinal lesions characteristic of necrotic enteritis in broiler chickens in the following treatment groups: INF A (chickens co-infected with Eimeria maxima and Clostridium perfringens and fed a commercial corn-based diet, n=27, SE: 0.095); INF B (chickens co-infected with E. maxima and C. perfringens and fed a PennHFD-based diet, n=24, SE: 0.092); CTL A (uninfected chickens fed a commercial corn-based diet, n=27); and CTL B (uninfected chickens fed a PennHFD-based diet, n=25). An incidence was counted if at least one area of necrosis (i.e., of any size) was found in the small intestine of the chicken.

FIG. 2 shows photographs of the flavonoid-rich corn cultivar PennHFD. The photo of the plants (left) was taken at the time of flowering. The photo of the corn cob (right) was taken at maturity.

### DETAILED DESCRIPTION

The present invention provides a novel flavonoid-rich corn cultivar designated PennHFD. Animal feeds comprising kernels of this corn and methods of using the animal feeds to reduce necrotic enteritis (NE) and improve growth performance in animals are also provided.

Market restrictions and consumer preference for antibiotic-free products have led the poultry industry to move towards antibiotic-free production. However, the discontinued use of antimicrobial growth promoters has led to an increased incidence of avian NE. In the present application, the inventors tested the ability of a flavonoid-rich corn cultivar, PennHFD, to serve as an alternative means to reduce NE. Using a chicken model of NE, they found that chickens fed a diet comprising PennHFD exhibited a lower incidence of NE intestinal lesions, higher body weight gain, lower feed conversion ratio, and lower mortality rates as compared to control chickens that were fed a diet comprising commercially available corn. Their data suggest that including high-flavonoid corn in the diet may improve the health and growth performance of both healthy birds and birds with NE.

### Corn:

In a first aspect, the present invention provides the novel corn cultivar PennHFD (ATCC Accession No: 202111002). The invention encompasses the seeds of this cultivar, plants grown from these seeds, and seeds produced by these plants. The invention further encompasses any corn plant having all of the physiological and morphological characteristics of corn cultivar PennHFD.

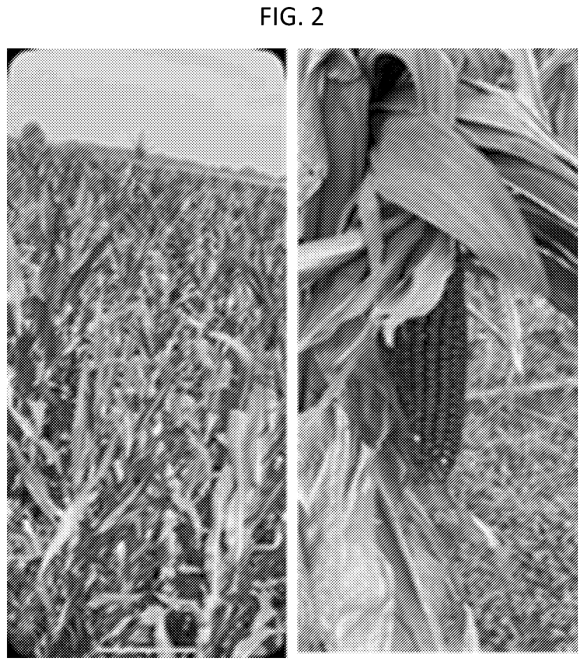

PennHFD is a flavonoid-rich corn cultivar. PennHFD plants contain high concentrations of flavonoids in their seeds and plant body but have a normal appearance (see FIG. 2).

Flavonoids are a class of polyphenolic secondary metabolites found in plants. They are powerful antioxidants with anti-inflammatory properties. As used herein, the term “flavonoid-rich” is used to describe corn that has higher flavonoid levels than those found in commercially available corn varieties, which contain low or undetectable levels of flavonoids. For example, PennHFD has a flavonoid concentration that is over 10 times (i.e., 11 times) higher than that of a commercially available corn line (see Examples).

As used herein, the term “plant” encompasses both whole plants and parts thereof, including cells, protoplasts, calli, clumps, embryos, pollen, ovules, flowers, glumes, panicles, leaves, stems, roots, root tips, anthers, pistils, and the like. This term also encompasses tissue cultures from which plants can be regenerated.

A “seed” is an embryonic plant enclosed in a protective outer covering along with a supply of food. The seeds of corn are also referred to as “kernels,” and these terms are used interchangeably herein. Seeds are the edible part of the corn plant and are commonly used as an energy source in animal feed, human food, and biofuel feedstock.

As used herein, the terms “cultivar” and “variety” are used interchangeably to refer to plants with the characteristics of a particular genotype or combination of different genotypes. Plants of a particular cultivar are distinguished from any other plant grouping by at least one characteristic.

### Animal Feed:

In a second aspect, the present invention provides animal feeds comprising seeds of the flavonoid-rich corn described herein. As used herein, the term “animal feed” refers to a product intended for consumption by an animal.

Suitable animals for which the feed may be formulated include, without limitation, agricultural animals, fish, birds, reptiles, companion animals, humans, and the like. As used herein, the term “agricultural animal” refers to any domesticated animal used to produce agricultural products. Agricultural animals include, without limitation, poultry, cows, pigs, sheep, and goats. In preferred embodiments, the feed is formulated for poultry, such as chickens or turkeys. Exemplary poultry feed compositions are described in the art (see, e.g., Poultry Nutrition 5th Edition, The Ray Ewing Co. Pasadena Calif. 1963). In some embodiments, the feed is formulated as described in Table 1. In some embodiments, the animal is suffering from or is at risk of developing NE or another infectious enteritis. In some embodiments, the animal is transmitting a food borne pathogen such as C. perfringens.

Animal feeds are often formulated differently for animals at different stages of development. For example, feeds formulated for layer chickens, broiler chickens, and chicks often include different ingredients to meet the distinct nutrient requirements of these developmental stages. Thus, in some embodiments, the feed is formulated for layer chickens. In other embodiments, the feed is formulated for broiler chickens.

The animal feeds may include key nutrients needed to meet the dietary requirements of a particular animal. The animal feeds comprise seeds of the flavonoid-rich corn described herein, which contribute to the carbohydrate component of the feed. The animal feed may further comprise carbohydrates from additional sources, a protein component, a fat component, or any combination thereof.

Common carbohydrate components found in animal feeds include, without limitation, cereal grains such as corn, wheat, sorghum, barley, rye, triticale, and oats.

Common protein components found in animal feeds include, without limitation, soybean, oilseed meals, legumes, abattoir, fishmeal, and meat processing by-products. Suitable oilseed meals include, without limitation, soybean, rapeseed/canola, sunflower, palm kernel, copra, linseed, peanut, and sesame seed meals.

Common fat components found in animal feeds include, without limitation, pig fat, beef fat, linseed oil, soy oil, sunflower oil, and palm oil.

The animal feed may comprise any commonly used feedstuff including, for example, silages, roots, tubers, fleshy fruits, grains, seeds, brewer's grains, pomace, brewer's yeast, distiller's spent grains, milling byproducts, byproducts of sugar production, starch and oil recovery, and various food wastes. The animal feed may also include feed additives such as antioxidants, amino acids, vitamins, and minerals (e.g., sodium, calcium, phosphorus, lysine, methionine, vitamin D3, niacin, vitamin B12, vitamin A, and riboflavin).

In some embodiments, the flavonoid-rich corn kernel constitutes about 25-60% of the feed, preferably 25%-35% of the feed. In the Examples, inventors used 31.53% corn in their feed. Thus, in some embodiments, the corn kernel constitutes about 31% of the feed.

The animal feed may be in the form of powder, pellet, grain, mash, or liquid. Corn is normally ground in a hammer mill and then is mixed with other ingredients to create the final diet, which is typically pelleted (e.g., for broiler chickens) or fed as mash (e.g., for layer chickens). Accordingly, the corn kernels may be ground or liquefied for use in the animal feed.

### Methods

In a third aspect, the present invention provides methods for reducing the incidence of NE in a population of animals. The methods comprise administering the corn kernels or an animal feed described herein to the animals.

Necrotic enteritis (NE) is an enteric disease of poultry characterized by patches of necrotic tissue on the intestinal epithelium. The causative agent of NE is the bacterium Clostridium perfringens (types A and C), a normal occupant of the chicken digestive tract. Intestinal lesions are indicative of this disease. Intestinal lesions may be classified according to a scoring system, such as the one described in the section of the Examples titled “Sampling and lesion scoring”. Other symptoms of NE include diarrhea, depression, ruffled feathers, aphagia, and mortality.

A “reduction in the incidence of NE” may be detected as a lower mortality rate, a lower incidence of intestinal lesions, a reduction in the growth of C. perfringens in the intestinal tract of the animal, or a reduction in any other symptom of this disease. Intestinal lesions can be detected, for example, by necropsy or by harvesting a piece of the intestine for histopathological analysis. Subclinical NE negatively impacts body weight gain and feed conversion ratio. Thus, a “reduction in the incidence of NE” may also be detected as an increase in body weight gain or a decrease in feed conversion ratio.

In some embodiments, a “reduction in the incidence of NE” comprises a reduction in the incidence of NE in the population of animals as compared to the incidence in a population of control animals. As used herein, the term “control animal” refers to a comparable animal (e.g., of the same breed, sex, and age) that was raised under the same or comparable conditions (e.g., diet, environment, etc.) but was fed a diet comprising commercially available corn rather than a diet comprising the flavonoid-rich corn described herein.

In the Examples, the inventors demonstrate that chickens fed a diet comprising flavonoid-rich PennHFD corn had lower mortality rates and lower incidence of intestinal lesions than control chickens fed a diet comprising commercially available corn following challenge with C. perfringens. Thus, in some embodiments, the methods result in a lower mortality rate, and, in some embodiments, the methods result in a lower incidence of intestinal lesions.

“Mortality rate” is a measure of the number of deaths in a particular population per unit of time, scaled to the size of that population. For example, mortality rate is commonly expressed in units of deaths per 1,000 individuals per year. The mortality rate of an animal population fed PennHFD corn according to the present methods may be at least 3%, 5%, 10%, 20%, 30%, 40%, or 50%, 60%, 70%, or 80% lower than the mortality rate of a population of control animals fed a diet comprising commercially available corn. Preferably, the mortality rate is at least 30%, 40%, 50%, or 60% lower than in the control population.

“Incidence of intestinal lesions” is the percentage of animals in the population that have detectable lesions in their intestines. The incidence of intestinal lesions in an animal population fed PennHFD corn according to the present methods may be at least 3%, 5%, 10%, 20%, 30%, 40%, 50%, 60%, 70%, or 80% lower than the incidence of intestinal lesions in a population of control animals fed a diet comprising commercially available corn. Preferably, the detectable lesion rate is at least 30%, 40%, 50%, or 60% lower than in the control population.

As used herein, “administering” may be carried out using any of the variety of procedures used to provide nutrients to animals. Suitable application methods include incorporating the PennHFD corn kernels into the feed of the animal or supplying the animal with a feed composition described herein. The kernels or feed may be supplied in accordance with the standard feeding practices of the farm for the animal.

In a fourth aspect, the present invention provides methods for improving the growth performance of an animal. The methods comprise administering the corn kernels or an animal feed described herein to the animal.

An “improvement in growth performance” may be detected as an increase in body weight gain, increase in feed intake, and/or a decrease in feed conversion ratio. In some embodiments, an “improvement in growth performance” comprises a better growth performance as compared to that of a control animal.

In the Examples, the inventors demonstrate that chickens fed a diet comprising flavonoid-rich PennHFD corn exhibited greater body weight gain and lower feed conversion ratios than control chickens fed a diet comprising commercially available corn. Importantly, this difference was seen in both the set of birds that were challenged with C. perfringens and as well as in birds that were not challenged with C. perfringens. Thus, in some embodiments, the methods result in greater body weight gain, and, in some embodiments, the methods result in a lower feed conversion ratio.

“Body weight gain (BWG)” is measured as the increase in animal weight during a particular period of time. The BWG of an animal fed PennHFD corn according to the present methods may be at least 3%, 5%, 10%, 20%, 30%, 40%, 50%, 60%, 70%, or 80% greater than the BWG of a control animal fed a diet comprising commercially available corn.

“Feed conversion ratio (FCR)” is the amount of feed that is required to produce a unit of meat. It is calculated by dividing the quantity of feed provided to the animal by the body weight gained by the animal. The FCR of an animal fed PennHFD corn according to the present methods may be at least 3%, 5%, 10%, 20%, 300%6, 40%, 50%, 60%, 70%, or 80% lower than the FCR of a control animal fed a diet comprising commercially available corn.

Suitable animals to which the methods for reducing the incidence of NE or the methods for improving growth performance may be applied include, without limitation, agricultural animals, fish, birds, reptiles, companion animals, humans, and the like.

In a fifth aspect, the present invention provides methods for producing flavonoid-rich corn plants. The methods comprise planting a plurality of PennHFD corn seeds under conditions favorable for the growth of corn plants. In some embodiments, the methods further comprise producing corn seed from the resulting corn plants and/or planting the corn seed. Methods for planting and growing corn and producing corn seed therefrom are well known in the art. PennHFD corn may be grown using any standard agronomic practices used to grow traditional or non-genetically modified corn.

The present invention encompasses all plants, or parts thereof, produced by the methods described herein, as well as the seeds produced by these plants. Further, any plants derived from corn cultivar PennHFD or produced from a cross using cultivar PennHFD are provided. This includes genetic variants, created either through traditional breeding methods or through transformation, as well as plants produced in a male-sterile form. Notably, this includes gene-converted plants developed by backcrossing. Any of the seeds, plants, or plant parts provided may be utilized for human food, livestock feed, or as a raw material in industry. Use of corn cultivar PennHFD in any plant breeding or transformation method is also encompassed by the present invention.

The inventors have demonstrated that high levels of flavonoids in the leaves of other flavonoid-producing corn cultivars deter fall armyworm feeding, resulting in reduced herbivory damage. See Chatterjee et al. (Journal of Pest Science, doi.org/10.1007/s10340-022-01535-y, 2022), which is hereby incorporated by reference in its entirety. Thus, in addition to their ability to reduce NE in poultry, the PennHFD corn plants disclosed herein may offer increased resistance to pests. Specifically, the seeds, corn stalks, and leaves of PennHFD all contain higher than normal amounts of flavonoids, which may deter pests, such fall armyworm and corn ear worm, and thereby prevent yield loss.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

Avian necrotic enteritis (NE) is an infectious disease that impacts poultry worldwide, causing economic losses. Discontinued use of antimicrobial growth promoters has been associated with high incidence of the disease and has created a need to find new therapeutic alternatives.

Flavonoids are polyphenolic compounds found in many plants that provide protection against environmental challenges and act as attractants for pollinators (Panche et al., 2016). Flavonoids have been shown to have several health-promoting properties in humans and animals, including anti-inflammatory and antibacterial activities (Cushnie and Lamb, 2011; Abotaleb et al., 2019; Farhadi et al., 2019; Jin, 2019; Maleki et al., 2019; Kopustinskiene et al., 2020). Flavonoids can modulate intracellular signaling pathways of both the innate and adaptive immune systems through several mechanisms of action, including inhibition of COX-2 activity and inhibition of the NF-κB and MAPK pathways (Chen et al., 2018). Flavonoids can act as antibacterial compounds by disrupting bacterial cell membranes, inhibiting the enzymes that support DNA replication, and inhibiting production of bacterial toxins (Górniak et al., 2019).

There is limited evidence suggesting that flavonoids can be used to treat NE. One study found that including a flavonoid-rich muscadine pomace additive in the diet of broiler chickens helped to partially ameliorate NE. However, muscadine pomace contains high concentrations of tannins that are known to negatively affect feed consumption in chickens, and this feed additive was found to reduce performance (McDougald et al., 2008). Thus, flavonoid-rich ingredients that do not negatively impact zootechnical parameters may be an effective means to control NE in poultry.

Corn (Zea mays L.) is a commonly used source of energy for poultry in the United States (Dei, 2017). Varieties of corn that have been genetically selected to contain higher concentrations of flavonoids have been shown to have antibacterial and anti-inflammatory properties (Nessa et al., 2012; Wu et al., 2020; Wu et al., 2021).

In view of the health-promoting effects of flavonoids and the importance of corn in poultry diets, the inventors hypothesized that a flavonoid-rich corn variety, referred to as PennHFD, could be used to ameliorate NE chickens. In the following example, the inventors compared the effect of a diet formulated with PennHFD to that of a diet based on commercially available corn in chickens subjected to a controlled NE challenge based on co-infection with Eimeria maxima and Clostridium perfringens. They found that chickens fed the PennHFD-based diet had a lower incidence of intestinal lesions (P=0.048), higher body weight gain (P<0.01), lower feed conversion ratio (P<0.01), and lower mortality rate (P=0.023) compared to birds fed the control diet. Therefore, the inclusion of high-flavonoid PennHFD corn in the diet of broiler chickens reduces the severity and negative impact of NE.

### Materials and Methods

### Development of the Corn Cultivar PennHFD

Parental plant materials were obtained from The Maize Genetics Cooperation Stock Center (maizecoop.cropsci.uiuc.edu). Parental materials included parent 1: maize inbred line 4Co63 (loci of interest: P1-ww, c1, b1, Pr1), parent 2: maize inbred line W22 (loci of interest: Pr1, A1, C1, R1), parent 3: genetic stock P1-rr4B2 (loci of interest: P1-rr, C1, R1, pr1), and parent 4: genetic stock MGS14273 (loci of interest: pr1, A1, A2, C1, R1). A description of loci implicated in the flavonoid biosynthesis pathway can be found in Sharma et al. (Genetics 188(1):69-79, 2011) and Sharma et al. (BMC Plant Biol 12:196, 2012), which are incorporated by reference in their entireties.

All genetic manipulations were performed using conventional plant breeding methods. The genetic composition of loci were determined using PCR-based genotyping and expression assays. Genetic stocks containing genes needed for increased accumulation of flavonoids in the desired plant tissues were crossed, and, in subsequent generations, selections were made to carry these genes forward. Specifically, the following crosses were made:
Single cross 1: parent 1×parent 2—F1 was backcrossed six times with parent 2 while selecting for purple aleurones/endosperms. This yielded BC6F1-Sc1.
Single cross 2: parent 3×parent 4—F1 was backcrossed six additional times with parent 4 while selecting for red pericarps of the seed. This yielded BC6F1-Sc2.
Double cross: BC6F1-Sc1×BC6F1-Sc2—F1 plants were genotyped via PCR and selected for flavonoid expression using spectrophotometric readings for two different classes of flavonoids. Another three cycles of backcrossing were performed using parent 3 (3 crosses) and parent 4 (3 crosses) to ensure that flavonoids were expressed in all parts of the seed. A final BC6Sc-Sc2 progeny was selfed to develop a series of inbred lines. One of the inbred lines yielded PennHFD-1.

### Experimental Design

The poultry experiment was conducted for 21 days using a completely randomized design with four treatments. A total of 400 day-old straight-run broiler chickens (Ross 308, Aviagen) were obtained from a local hatchery (Belleville, PA). Upon arrival, the birds were randomly allocated into 20 floor pens (2.6 m²). The pens were randomly selected to receive one of the following treatments: CTL A (uninfected birds fed a commercial corn-based diet); CTL B (uninfected birds fed a PennHFD-based diet); INF A (birds co-infected with Eimeria maxima and Clostridium perfringens and fed a commercial corn-based diet); and INF B (birds co-infected with Eimeria maxima and Clostridium perfringens and fed a PennHFD-based diet). Infected and non-infected controls were held in adjacent but identical rooms to avoid cross-contamination. All pens were equipped with a manual self-feeder and automatic nipple drinkers, and the birds had ad libitum access to feed and water. The brooding conditions were adjusted throughout the experiment based on recommendations for the genetic line (Aviagen, 2018).

### Diet Formulation

Two diets were formulated to include ingredients reported to be important predisposing factors for NE in broiler chickens: wheat and fishmeal (Prescott et al., 2016) (Table 1). The diets were identical, except for the corn variety. “Feed A” was formulated with a commercially available corn and “Feed B” was formulated with PennHFD, a proprietary corn variety that is known to be rich in flavonoids. The diets were formulated to meet or exceed the dietary requirements for broiler chickens set by the National Research Council (1994).

To compare the flavonoid content of the PennHFD corn and the commercially available corn, the relative concentration of flavylium ion (i.e., a product derived from flavonoids during the extraction process) was measured as previously described (Grotewold et al., 1998; Wu et al., 2021). Briefly, 100 mg of ground kernel was incubated in 1 mL of acidic butanol (HCL:butanol=3:7, v/v) at 37° C. for 1 hour. The samples were centrifuged for 20 seconds at 10,000×g and the supernatant was removed. The supernatant was analyzed by spectrophotometry with a Cytation3 microplate reader (BioTek, Winooski, VT). Absorbance was taken at 550 nm, and the relative concentration was expressed as absorbance per gram of plant tissue.

Nutrients from both corn types were analyzed by proximate analysis performed at a third-party laboratory (Cumberland Valley Analytical Services, Waynesboro, PA; Table 2).

TABLE 1
Composition of “Feed A” and “Feed B”

| Ingredient | Composition (%) |
|---|---|
| Corn | 31.53 |
| Wheat | 30.00 |
| Soybean meal | 20.20 |
| Fish meal | 15.00 |
| Lard | 2.57 |
| Vitamin and mineral premix | 0.40 |
| Salt | 0.30 |

*Different sources of corn were used in the two diets: Feed A contained commercial corn, while Feed B contained the high-flavonoid corn PennHFD.

TABLE 2
Composition analysis of the two corn varieties

| Nutrient | Commercial corn | PennHFD |
|---|---|---|
| Moisture | 8.8 | 9.1 |
| Dry matter | 91.2 | 90.9 |
| Crude protein | 13.2 | 12.9 |
| Lignin (% DM) | 2.02 | 2.23 |
| Crude fat (% DM) | 4.03 | 4.29 |
| Ash (% DM) | 1.85 | 2.07 |
| Calcium (% DM) | 0.01 | 0.02 |
| Phosphorus (% DM) | 0.33 | 0.40 |
| Magnesium (% DM) | 0.11 | 0.15 |
| Potassium (% DM) | 0.40 | 0.35 |
| Sodium (% DM) | 0.01 | 0.01 |
| Iron (ppm) | 31 | 95 |
| Manganese (ppm) | 10 | 17 |
| Zinc (ppm) | 26 | 27 |
| Copper (ppm) | 2 | 4 |
| Metabolizable energy (Mcal/kg) | 3.35 | 3.28 |

*% DM: percent dry matter

### Necrotic Enteritis Model

Chicks at 13 days of age were infected by oral gavage with 5,000 Eimeria maxima oocysts. Aliquots of three strains of C. perfringens (two NetB positive and one NetB negative) that were isolated from field cases of NE were inoculated into fluid thioglycolate medium (Neogen®, Lansing, MI) and incubated anaerobically at 37° C. for 24 hours to reach a final inoculum concentration of 1×10⁹ CFU/mL. Anaerobiosis was achieved using the AnaeroPack® System (Mitsubishi Gas Chemical America, New York, NY).

Twelve hours before the first inoculation with C. perfringens, feed from all pens was removed. On days 18 and 19, the feed of all pens in the infected treatments was inoculated with 1 mL of 1×10⁹ CFU of C. perfringens per bird.

### Growth Performance and Mortality

On day 21, all remaining animals and feed were weighed to calculate feed consumption (day 0-21), total body weight gain (day 0-21), and total feed conversion ratio (day 0-21). Mortality data was recorded throughout the experiment.

### Sampling and Lesion Scoring

On day 21, 103 birds (˜five birds/pen) were randomly selected for evaluation of intestinal lesions by a trained individual that was blinded to treatment. The animals were euthanized by cervical dislocation, weighed, and necropsied.

Lesions were classified according to a scoring system, ranging from 0 to 5 (Lorenzoni et al., 2019), modified from Gholamiandehkordi et al. (2007). Briefly, a score of 0 was assigned to birds with no sign of lesions in their intestines. A score of 1 was assigned to birds with one or two isolated areas of necrosis smaller than 3 mm in diameter. A score of 2 was assigned to birds with one or two isolated patches of necrosis of 3-10 mm in diameter. A score of 3 was assigned to the birds with 3 or more necrotic patches along the length of the small intestine. A score of 4 was assigned to birds with more than 3 necrotic patches in close succession covering at least 10 cm of the intestine. A score of 5 was assigned to birds with necrotic patches that fused together, covering at least 10 cm of the intestine.

### Statistical Analysis

Statistical analyses were performed using the software Minitab 19® (Minitab Inc., State College, PA). To test the effects of diet and infection on body weight gain (BWG), feed conversion ratio (FCR), and mortality, a general linear model procedure was used. Pen served as the experimental unit and diet (Feed A and Feed B) and group (infected and control) were assumed to be fixed effects. Optimal box-cox transformations were made for BWG and FCR A statistical difference was claimed when P≤0.05, and pairwise comparisons were performed using a Fisher least significant difference (LSD) test.

The incidence of intestinal lesions was compared between the two infected treatments (INF A and INF B) using a Z-test for comparing two proportions, and a difference was claimed when P≤0.05. Since none of the animals sampled in the control treatments (CTL A and CTL B) presented lesions, the difference between these treatments was not tested.

Ordinal logistic regression was carried out to test the severity of lesions based on the intestinal lesion scores. The two different diets were used as categorical predictors with Feed A used as the reference group. Significance was determined when P≤0.05.

### Results

### Feed Consumption, Body Weight Gain, Feed Conversion Ratio, and Mortality

The mean feed consumption, total body weight gain, total feed conversion ratio, and mortality of each treatment group is shown in Table 3. There was a difference (P<0.01) in the mean body weight gain between Feed A and Feed B. Birds from the treatments INF A and CTL A showed lower body weight gain than birds from the treatments INF B and CTL B, respectively. The mean feed conversion ratios were also different (P<0.01). Birds in the treatments INF A and CTL A had higher feed conversion ratios compared to birds in the treatments INF B and CTL B, respectively. In addition, mortality rate means were different (P=0.023). As expected, the pairwise comparison did not show a difference in mortality between the treatments CTL A and CTL B. However, birds from the treatment INF B had 42.86% less mortality compared to birds from the treatment INF A.

TABLE 3
Mean feed consumption (FC; kg), total body weight gain (BWG; kg), total feed
conversion ratio (FCR), and mortality of broilers fed commercial corn (Feed
A) or PennHFD corn (Feed B) with (infected) or without (control) NE challenge.

| | Control | | | | Infected | | | | |
|---|---|---|---|---|---|---|---|---|---|
| | Feed A | | Feed B | | Feed A | | Feed B | | P- |
| | Mean | S.E. | Mean | S.E. | Mean | S.E. | Mean | S.E. | value |
| FC | 24.520^{a} | 0.412 | 24.796^{a} | 0.190 | 19.223^{b} | 0.655 | 20.729^{b} | 0.385 | 0.463 |
| BWG | 19.483^{B} | 274 | 20.519^{A} | 0.143 | 9.320^{D} | 1.135 | 13.293^{C} | 0.643 | <0.01 |
| FCR | 1.253^{C} | 0.012 | 1.208^{D} | 0.004 | 1.470^{A} | 0.026 | 1.400^{B} | 0.024 | <0.01 |
| Mortality | 0.040^{bc} | 0.018 | 0.000^{c} | 0.000 | 0.375^{a} | 0.083 | 0.161^{b} | 0.042 | 0.023 |

Different superscripts in the same row denote statistically significant differences (P≤0.05).
Lowercase superscripts (^{abc}) indicate P≤0.05 and uppercase superscripts (^{ABCD}) indicate P≤0.01. First alphabetical letters indicate the largest mean in the row.
Infected birds were co-infected with E. maxima and C. perfringens.

### Lesions

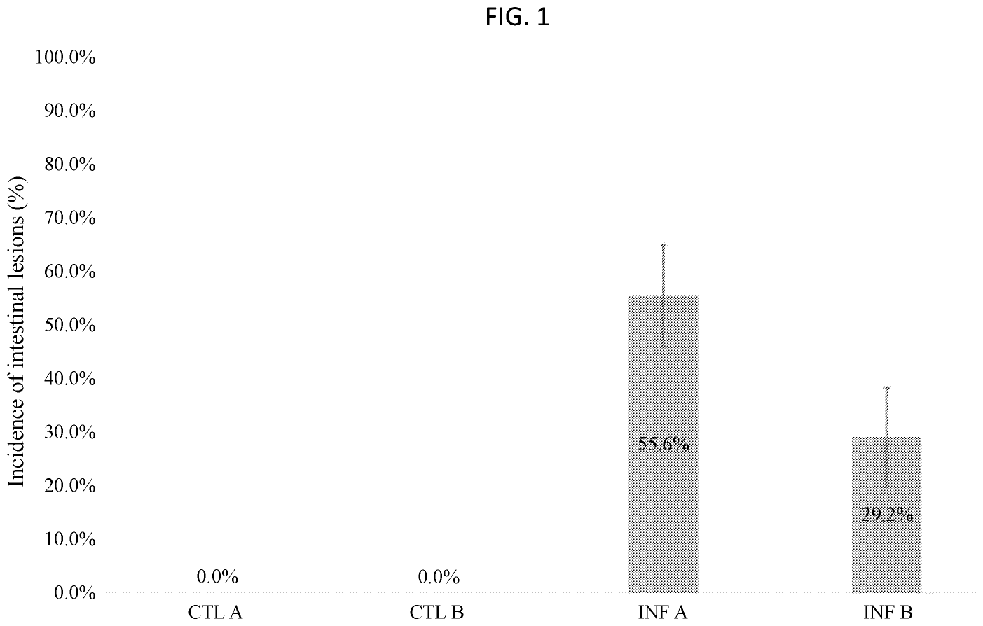

The incidence of NE lesions is presented in FIG. 1. As expected, no necrotic lesions were found in the intestines of the 52 birds sampled from the control treatments (CTL A and CTL B). In the infected treatments, 43.13% (22 of 51) of the sampled birds presented lesions characteristic of NE. Birds from the treatment INF A had a lesion incidence of 55.55%, while birds from the treatment INF B had a lesion incidence of 29.16% (P=0.048). There was no difference in the severity of NE lesions (lesion scores) between the treatments INF A and INF B.

### Proximate Analysis and Flavylium Ion Concentration

The proximate analysis indicated that the commercial corn line contained 13.2% crude protein and 3,351 kcal/kg of metabolizable energy. The PennHFD corn line contained 12.9% crude protein and 3,285 kcal/kg of metabolizable energy (Table 2). The relative flavylium ion concentration, which was measured at 550 nm and is representative of total flavonoid concentration, was 1.98 absorbance/g for the PennHFD and 0.17 absorbance/g for the commercial corn line.

### DISCUSSION

The use of antibiotics as growth promoters has been steadily decreasing in poultry production (Brewer and Rojas, 2008; Millet and Maertens, 2011) and alternatives such as flavonoids have been presented as a potential replacement for these compounds. In our experiment, a flavonoid-rich corn line reduced mortality and the presence of lesions indicative of NE in broiler chickens. To our knowledge, this is the first study that has tested the effects of a high-flavonoid corn variety on broilers subjected to NE.

Flavonoids can be found in many higher plants and have antibacterial and anti-inflammatory properties (Farhadi et al., 2019; Maleki et al., 2019). For example, McDougald et al. (2008) studied the effects of including 0.5% or 2% muscadine pomace, a flavonoid-rich in-feed additive derived from the production of wine, in broiler chickens subjected to NE. When compared to a diet without additives, birds consuming muscadine pomace had lower mortality, lower lesion scores, and improved feed conversion. However, the addition of muscadine pomace affected feed intake, reduced body weight, and increased the feed conversion of the birds (McDougald et al., 2008). This is likely due to the high concentration of tannins in muscadine pomace, which are known to limit feed consumption and affect the productive performance of birds (Chung et al., 1998). Since corn can be included at a high rate in the diets of animals, lines of corn with high concentrations of flavonoids may be an ideal alternative for the control of intestinal diseases in livestock production.

In our experiment, birds fed a PennHFD-based diet and challenged with C. perfringens (INF B) had decreased mortality rates, decreased incidence of intestinal lesions, and improved body weight gain and feed conversion ratio compared to challenged birds receiving the control diet (INF A). These results are consistent with previous studies that observed an increased growth performance and a reduction in mortality and C. perfringens counts after the inclusion of phytogenic in-feed additives in birds subjected to NE (Granstad et al., 2020). In contrast, Leusink et al. (2010) did not see an effect on growth performance and mortality in broilers challenged with NE after including up to 0.016% of a flavonoid-rich cranberry fruit extract in the diet. This may indicate that the active compound was not included in a sufficient amount to produce the desired effects in the challenged birds.

Lines of corn that have been genetically selected to have high flavonoid contents have shown important anti-inflammatory activity in vitro and in vivo in mice subjected to intestinal inflammation (Wu et al., 2020; Wu et al., 2021). Factors that promote intestinal inflammation have been correlated with decreased animal performance, multiplication of C. perfringens, and development of NE (Timbermont et al., 2011). The diets used in our experiment contain ingredients reported to induce inflammation, such as wheat and fishmeal (Branton et al., 1987; Prescott et al., 2016). The uninfected birds that received high flavonoid corn (CTL B) had improved body weight gain and feed conversion ratio, which could be a result of the reduction in subclinical intestinal inflammation. This agrees with reports showing that flavonoid-rich feed additives can improve the growth performance and immunity of birds in the absence of clinical disease (Zhou et al., 2019).

It is important to underscore that PennHFD has a lower percentage of crude protein (12.9%) and lower metabolizable energy (3,285 kcal/kg) compared to the commercial line (13.2% and 3,351 kcal/kg), which suggests that the improved growth performance achieved with PennHFD is not a result of these nutritional aspects.

Although the mechanisms of action that allow the high-flavonoid corn to ameliorate NE in chickens were not investigated in our study, we speculate that the anti-inflammatory and antibacterial properties of flavonoids could play a key role in the control of this disease.

In conclusion, the inclusion of a flavonoid-rich corn in the diets of broiler chickens subjected to experimental NE resulted in reduced mortality, reduced incidence of intestinal lesions, and improved growth performance. In addition, birds that were not challenged with NE also had improved growth performance when fed a high-flavonoid corn. Therefore, high-flavonoid corn may serve as a potential alternative for improving health and performance in the absence of antimicrobials in birds challenged with NE or undergoing subclinical enteritis.

### REFERENCES

Abotaleb, M., Samuel, S. M., Varghese, E., Varghese, S., Kubatka, P., Liskova, A., Büsselberg, D. 2019. Flavonoids in cancer and apoptosis. Cancers (Basel). 11:1-39.
Adhikari, P., Kiess, A., Adhikari, R., Jha, R 2020. An approach to alternative strategies to control avian coccidiosis and necrotic enteritis. J. Appl. Poult. Res. 29:515-534.
Al-Sheikhly, F., Al-Saieg, A. 1980. Role of coccidia in the occurrence of Necrotic Enteritis of chickens. Avian Dis. 24:324-333.
Aviagen. 2018. Ross Management Book. Aviagen. Accessed January 2021. p. 1-144. https://en.aviagen.com/assets/Tech_Center/Ross_Broiler/Ross-BroilerHandbook2018-EN.pdf
Branton, S. L., Reece, F. N., Hagler, W. M. 1987. Influence of a wheat diet on mortality of broiler chickens associated with necrotic enteritis. Poult. Sci. 66:1326-1330. doi:10.3382/ps.0661326
Brewer, M. S., Rojas, M. 2008. Consumer attitudes toward food safety issues. J. Food Saf. 28:1-22.
Broom, L. J. 2017. Necrotic enteritis; Current knowledge and diet-related mitigation. Worlds Poult. Sci. J. 73:281-292.
Caly, D. L., D'Inca, R., Auclair, E., Drider, D. 2015. Alternatives to antibiotics to prevent necrotic enteritis in broiler chickens: A microbiologist's perspective. Front. Microbiol. 6:1-12.
Chen, L., Teng, H., Jia, Z., Battino, M., Miron, A., Yu, Z., Cao, H., Xiao, J. 2018. Intracellular signaling pathways of inflammation modulated by dietary flavonoids: the most recent evidence. Crit. Rev. Food Sci. Nutr. 58:2908-2924.
Chung, K.-T., Wei, C.-I. 1998. Are tannins a double-edged sword in biology and health? Trends in Food Science & Technology. 9:168-175.
Cushnie, T. P. T., Lamb, A. Recent advances in understanding the antibacterial properties of flavonoids. Int. J. Antimicrob. Agents. 38:99-107.
Dei, H. K. 2017. Assessment of Maize (Zea mays) as feed resource for poultry. Pages 1-30 in Poultry Science. ed. Manafi, M., Rijeka, Croatia.
Farhadi, F., Khameneh, B., Iranshahi, M., Iranshahy, M. 2019. Antibacterial activity of flavonoids and their structure-activity relationship: An update review. Phyther Res. 33:13-40.
Gholamiandehkordi, A. R., Timbermont, L., Lanckriet, A., Broeck, W. V. D., Pedersen, K., Dewulf, J., Pasmans, F., Haesebrouck, F., Ducatelle, R, Immersel, F. V. 2007. Quantification of gut lesions in a subclinical necrotic enteritis model. Avian Pathol. 36:375-382.
Górniak, I., and Bartoszewski, R 2019. Comprehensive review of antimicrobial activities of plant flavonoids. Phytochem Reviews. 18:241-272.
Granstad, S., Kristoffersen, A. B., Benestad, S. L., Sjurseth, S. K., David, B., Serensen, L., Fjermedal, A., Edvardsen, D. H., Sanson, G., Lovland, A., Kaldhusdal, M. 2020. Effect of feed additives as alternatives to in-feed antimicrobials on production performance and intestinal Clostridium perfringens counts in broiler chickens. Animals. 10:1-19.
   Grotewold, E., M. Chamberlin, M. Snook, B. Siame, L. Butler, J. Swenson, S. Maddock, G. St. Clair, and B. Bowen. 1998. Engineering secondary metabolism in maize cells by ectopic expression of transcription factors. Plant Cell 10:721-740.
Jin, Y. S. 2019. Recent advances in natural antifungal flavonoids and their derivatives. Bioorganic Med. Chem. Lett. 29:1-13. doi:10.1016/j.bmcl.2019.07.048
Kaldhusdal, M., Benestad, S. L., Levland, A. 2016. Epidemiologic aspects of necrotic enteritis in broiler chickens—disease occurrence and production performance. Avian Pathol. 45:271-274.
Kopustinskiene, D. M., Jakstas, V., Savickas, A., Bernatoniene, J. 2020. Flavonoids as anticancer agents. Nutrients. 12:1-25.
Leusink, G., Rempel, H., Skura, B., Berkyto, M., White, W., Yang, Y., Rhee, J. Y., Xuan, S. Y., Chiu, S., Silversides, F., Fitzpatrick, S., Diarra, M. 2010. Growth performance, meat quality, and gut microflora of broiler chickens fed with cranberry extract. Poult. Sci. 89:1514-1523. doi:10.3382/ps.2009-00364
Lorenzoni, A. G., Rojas-Núñez, I., Moore, A. F. 2019. Effect of Aspirin on the Intestinal Response to a Necrotic Enteritis Challenge. Avian Dis. 63:686-692.
Maleki, S. J., Crespo, J. F., Cabanillas, B. 2019. Anti-inflammatory effects of flavonoids. Food Chem. 299:1-11.
McDougald, L. R., Hofacre, E. C., Mathis, G., Fuller, L., Hargrove, J. L., Greenspan, P., Hardle, D. K. 2008. Enhancement of resistance to coccidiosis and necrotic enteritis in broiler chickens by dietary Muscadine pomace. Avian Dis. 52:646-651.
Millet, S., Maertens, L. 2011. The European ban on antibiotic growth promoters in animal feed: From challenges to opportunities. Veterinary Journal. 187:143-144.
Nessa, F., Ismail, Z., Mohamed, N. 2012. Antimicrobial Activities of Extracts and Flavonoid Glycosides of Corn Silk (Zea mays L). Int. J. Biotechnol. Wellness Ind. 1:115-121.
Panche, A. N., Diwan, A. D., Chandra, S. R. 2016. Flavonoids: an overview. J. Nutr. Sci. 5:1-15.
Prescott, J. F., Parreira, V. R., Gohari, I. M., Lepp, D., Gong, J. 2016a. The pathogenesis of necrotic enteritis in chickens: what we know and what we need to know: a review. Avian Pathol. 45:288-294.
Prescott, J. F., Smyth, J. A., Shojadoost, B., Vince, A. 2016b. Experimental reproduction of necrotic enteritis in chickens: a review. Avian Pathol. 45:317-322.
Timbermont, L., Haesebrouck, F., Ducatelle, R., Immerseel F. V. 2011. Necrotic enteritis in broilers: An updated review on the pathogenesis. Avian Pathol. 40:341-347.
Tsiouris, V., Georgopoulou, I., Batzios, C., Pappaioannou, N., Ducatelle, R., Fortomaris, P. 2015 High stocking density as a predisposing factor for necrotic enteritis in broiler chicks. Avian Pathol. 44:59-66. doi:10.1080/03079457.2014.1000820
Wade, B., Keyburn, A. 2015. The true cost of necrotic enteritis. Poultry World. Accessed May. 2021. https://www.poultryworld.net/Meat/Articles/2015/10/The-true-cost-of-necrotic-enteritis-2699819W/.
Wu, B., Bhatnagar, R., Indukuri, V. V., Chopra, S., March, K., Cordero, N., Chopra, S., Reddivari, L. 2020. Intestinal mucosal barrier function restoration in mice by maize diet containing enriched Flavan-4-Ols. Nutrients. 12:1-13.
Wu, B., Chang, H., Marini, R., Chopra, S., Reddivari, L. 2021. Characterization of maize near-isogenic lines with enhanced flavonoid expression to be used as tools in diet-health complexity. Front. Plant. Sci. 11:1-13. doi:10.3389/fpls.2020.619598.
Zhou, Y., Mao, S., Zhou, M. 2019. Effect of the flavonoid baicalein as a feed additive on the growth performance, immunity, and antioxidant capacity of broiler chickens. Poult. Sci. 98:2790-2799. doi:10.3382/ps/pez071

### DEPOSIT INFORMATION

A deposit of the Pennsylvania State University proprietary corn cultivar PennHFD disclosed above and recited in the appended claims has been made with the American Type Culture Collection (ATCC), 10801 University Boulevard, Manassas, Va. 20110. The date of deposit was Nov. 5, 2021. The deposit was found viable on Nov. 10, 2021. All restrictions will be irrevocably removed upon granting of a patent, and the deposit is intended to meet all of the requirements of 37 C.F.R. §§ 1.801-1.809. The ATCC Accession Number is 202111002. The deposit will be maintained in the depository for a period of thirty years, or five years after the last request, or for the enforceable life of the patent, whichever is longer, and will be replaced as necessary during that period.

We claim:

1. A corn seed of the cultivar PennHFD, a representative sample of seed of said cultivar having been deposited under ATCC Accession No 202111002.

2. A corn plant, or a part thereof, produced by growing the seed of claim 1.

3. A corn plant, or a part thereof, having all of the physiological and morphological characteristics of the corn plant of claim 2.

4. A corn seed produced by the corn plant of claim 2.

5. An animal feed comprising the corn seed of claim 1.

6. The animal feed of claim 5, wherein the corn seed constitutes about 25-80% of the feed, preferably 25-50%, more preferably 25%-35%.

7. The animal feed of claim 5 further comprising one or more ingredient selected from the group consisting of: wheat, sorghum, barley, rye, triticale, oats, soybean meal, an oilseed meal, legumes, abattoir, fishmeal, meat processing by-products, pig fat, beef fat, linseed oil, soy oil, sunflower oil, and palm oil.

8. The animal feed of claim 7, wherein the feed is formulated as described in Table 1.

9. The animal feed of claim 5, wherein the animal feed is formulated for poultry.

10. The animal feed of claim 9, wherein the animal feed is formulated for layer chickens or broiler chickens.

11. A method for reducing the incidence of necrotic enteritis in a population of animals, the method comprising: administering the corn seed of claim 1, or an animal feed made therefrom, to the animals.

12. The method of claim 11, wherein the method results in a lower mortality rate.

13. The method of claim 11, wherein the method results in a lower incidence of intestinal lesions.

14. A method for improving the growth performance of an animal, the method comprising: administering the corn seed of claim 1, or an animal feed made therefrom, to the animal.

15. The method of claim 14, wherein the method results in greater body weight gain.

16. The method of claim 14, wherein the method results in a lower feed conversion ratio.

17. A method for producing flavonoid-rich corn plants, said method comprising planting a plurality of corn seeds of claim 1 under conditions favorable for the growth of corn plants.

18. The method of claim 17 further comprising producing corn seed from the resulting corn plants.

19. A corn seed produced by the method of claim 18.
